(19) United States
(12) Patent Application Publication
(10) Pub. No.: US 2026/0351358 A1
(43) Pub. Date: Nov. 5, 2026

(54) SYSTEM, METHOD AND PRODUCT TO MEASURE THE SUBSTANCE LEVELS FROM THE EEG DATA AND TO IMPROVE THE ACCURACY OF MEASURING LEVELS OF SUBSTANCE(S) FROM THE EEG DATA

(71) Applicant: Krishna GANDHI, Middlesex (GB)

(72) Inventor: Krishna GANDHI, Middlesex (GB)

(21) Appl. No.: 18/833,371

(22) PCT Filed: Jan. 30, 2023
(86) PCT No.: PCT/IB2023/050795

§ 371 Date: Jul. 25, 2024

(30) Foreign Application Priority Data
Jan. 28, 2022 (IN) 202211004922

(51) Int. Cl. G16H 10/40; A61B 5/374; A61B 5/398; G16H 10/60

(52) U.S. Cl. CPC G16H 10/40; A61B 5/374; A61B 5/398; G16H 10/60

(57) ABSTRACT

Methods, Apparatus, Systems and Devices enabled to improve the accuracy of measuring levels of substance(s) from the EEG data by taking into account data parameters. The data parameters are selected from either age, gender or sleep-wake state or for gender and sleep-wake state, or any combination thereof resulting in more than one EEG algorithm per substance. Further refining process involve application of General Linear Models and Non-Linear models for certain steps, use of AI/ML and Auto-regression and feedback loops to derive specialised and specific variables for each substance and with brain dynamics consideration. A serviceable system to apply the outcomes of the methods to measure levels of substance(s) from the EEG data are implemented in a SAAS system or as a digital based service.

### FIELD OF THE INVENTION

Embodiments of the present invention relates information technology and health computer applications. More particularly, the present application relates to methods, apparatus and systems for measuring the levels of Hormones, Neurotransmitters, Neuromodulators, Bio Markers, EEG Digital Markers or the like.

### BACKGROUND OF THE INVENTION

### Description of the Related Art

In 2007 a small study revealed the Electroencephalograph (EEG) had a significant capacity to estimate the levels of salivary cortisol later published in a GB patent as a Method and apparatus for measuring the levels of hormones or neuro transmitters and in patent WO2013008011A1. Previous papers referenced in WO2013008011A1 demonstrated the changes in the EEG to indicate stress and cortisol levels as well as the relationship between the EEG and another hormone, Testosterone.

The study published in WO2013008011A1 took it one step further to extract a quantified level of cortisol instead of the categorical high and low levels shared in the references of Gandhi WO2013008011A1.

The conclusion was that the EEG had the capacity to measure more than cortisol in the saliva, as the essential features applied for estimating the hormones, biomarkers and neurotransmitters levels in the blood, urine or any other method in the body are found in numerous scientific journals, books and papers.

These essential features in summary included average power of EEG frequency bands showing a correlation with increasing or decreasing levels of a substance or a correlation with a biomarker from at least two EEG signals.

WO2013008011A1 also mentions methods to further improve the accuracy of the essential feature and that is to correlate the frequency in to smaller band bins (e.g. 1 Hz bins) with substance levels to find the precise frequency bands that correlates with the levels of the substance.

Quantitative EEG (qEEG) techniques include the computation of power and associated scalp topographic maps for given frequency bands. Such techniques have been used to illustrate, diagnose and investigate neuropsychological states such as depression, alcoholism, schizophrenia or cognitive functions such as attention disorders, memory and vastly used to diagnose epilepsy. Cortisol is linked to the circadian rhythm where the levels are low during sleep to rise to a peak on wake known as the cortisol basal level. Cortisol activity and levels are involved in the regulation of this rhythm. The EEG data during sleep and wake is also distinguishable. The brain operates differently in both sleep and in wake. In sleep the thalamus cortical network changes significantly which results in a different operational state.

Both sleep-wake and gender groups demonstrate different operations which also correlate to significantly different activity between hormones and neurotransmitters which provides the basis to run the data analyses by either gender, sleep-wake-state or both to optimise the algorithm to measure or provide an indication of the level of substance required to measure.

Therefore, in light of the foregoing discussion, there exists a need to overcome the aforementioned drawbacks of the conventional devices and methods for improving the accuracy of measuring levels of substance(s) from the EEG.

Accordingly, the present invention is able to improve the accuracy of measuring levels of hormones, and neurotransmitters, neuromodulators and or biomarkers of at least one substance from the EEG data based on data parameters, applying General Linear Models, such as Ordinary Least-Squared (OLS), auto regression, non-linear models for specifying essential features, optimising of smaller steps and feeding more refined variable into the General Linear Models.

As a result the present invention can derive EEG algorithms at scale for many multiple substances through an automate pipeline between processes of FIG. 1 and FIG. 2.

The improved algorithms are used to update the present inventions of a serviceable system to measure substance levels from the EEG data.

### SUMMARY OF THE INVENTION

Various embodiments of present invention disclose various systems, components, modules, apparatus, wearable device and methods related to improving the accuracy to determine at least one substance level in a body of a subject. Further, the present invention is able to analyse a non-invasive measurement technique for measuring the levels of substance selected from hormones, neurotransmitters, neuromodulators, biomarkers, EEG digital markers of hormones, neurotransmitters, neuromodulators and biomarkers in the body based on a plurality of data parameters.

A study was carried out to further validate the capacity of the EEG to measure substance levels in the body from any medium. The present invention discloses the findings for the EEG's capacity to measure/estimate/predict the blood hormones levels of: Cortisol, ACTH, Estrogen and Testosterone. The results of the 2015 study can be seen in FIG. 9A-9F and carried out at a sleep research institute using clinical grade EEG with equal males and females.

To test the extent of the outcomes of these EEG algorithms derived for Cortisol, ACTH, Estrogen and Testosterone, a one-day trial study was setup using an EEG wearable with two new subject participants. The results of this one-day trial FIG. 9G-9L demonstrates the case for the present invention to be a reliable screening tool based on improvements in the method to derive the EEG algorithm.

Embodiments of the present invention include methods to improve the accuracy of deriving the EEG algorithm for substances such as the steps of obtaining and analysing data to determine at least one average power for each of a plurality of predetermined frequency bands, performing a linear algebra operation on the at least one data parameter, such as gender, sleep-wake state and age-group based on at least three average powers to model a relationship between an actual level of said at least one substance level and a predicted level of the at least one substance level, applying a trained artificial intelligence (AI) model that is adapted to analyze collected data and modeled relationship to determine the at least one substance level in the body, comparing the results that are statistically significant to determine the at least one substance level in the body of the subject, and select any one significant result that is reasonable to determine the at least one substance level.

Embodiments of the present invention, disclose an improved system of determining a substance level in a body of a subject from data in a regulatory network management. The system includes at least one user device, a communication network, a user identification module and a signal processing module. The one or more user devices and at least one remote server is in communication with the user identification module, the communication network for individual users is connected via the at least one user device, and a signal processing module configured to obtain an average power for each of a plurality of predetermined frequency bands.

Further, the at least one user device is operable to authorize sharing of selected at least one data parameter with a third-party device via a graphical user interface (GUI), thereby creating selected permissible data, and the user identification module is operable to exchange the selected permissible data with said third-party device.

In one embodiment, the at least one substance level determined is hormones, neurotransmitters, neuromodulators, biomarkers, EEG digital markers of hormones, neurotransmitters, neuromodulators and biomarkers.

In one embodiment, the method further includes the steps of: collecting at least one data parameter to determine data, evaluating a value from said at least one average power derived for each frequency band, and wherein said value is evaluated by combining said at least one average power for each frequency band by dividing and/or multiplying according to a predetermined order, and wherein said predetermined order is determined based on said at least one substance level, obtaining an estimate of said at least one substance level from an equation: Y=bX+C, and wherein Y is said at least one substance level to be predicted, X is value and b and C are constants, wherein said value of X is a amalgamated ratio obtained by combining said at least one average power for said each frequency band by dividing and/or multiplying according to a predetermined order; and wherein said plurality of predetermined frequency bands are frequency bands which have a correlation with increasing or decreasing levels of said at least one substance level to be measured.

In accordance with an aspect of the embodiments, the one or more user characteristics is selected from birth gender of said individual users, age, sleep state and sleep stages.

In further embodiment, the method further includes the steps of: determining the predetermined frequency bands by measuring said average power for said plurality of frequency bands for at least one substance level of each substance and selecting at least one band which illustrates a correlation with increasing or decreasing order of substance levels, calculating a corresponding set of average power values of the predetermined frequency bands for at least three substance levels for each substance, plotting a plurality of calculated values for each substance level against each substance; and selecting a ratio which provides a plot closest to a straight line wherein, varying boundaries of the predetermined frequency bands to obtain a better correlation to a straight line.

In yet another embodiment of the present invention, the method further comprises the steps of determining the predetermined frequency bands by measuring the average power for the plurality of frequency bands for at least one substance level of each substance and selecting at least one band which illustrates a correlation with increasing or decreasing order of substance levels, calculating a corresponding set of average power values of the predetermined frequency bands for at least three substance levels for each substance, plotting a plurality of calculated values for each substance level against each substance, and selecting a ratio which provides a plot closest to a straight line wherein, varying boundaries of said predetermined frequency bands to obtain a better correlation to a straight line.

In yet another embodiment of the present invention, the method further comprises the steps of mounting at least one wearable device on a user's head, or contact with user skin to measure said at least one data parameter, measuring EEG data with said at least one wearable device, and wherein said EEG data is collected from at least EEG signals and receiving EEG data from said at least one wearable device via said communication network, and measuring EOG data with said at least one wearable device, and wherein said EOG data is collected from at least EEG signals and receiving EOG data from said at least one wearable device via the communication network.

In one embodiment, the data is selected from the EEG data and the EOG data.

In one embodiment, the system further includes an estimation module configured to obtain an estimate of said at least one substance level from an equation: Y=bX+C, and wherein Y is said at least one substance level to be predicted, X is value and b and C are constants; wherein said value of X is a ratio obtained by combining said at least one average power for said each frequency band by dividing and/or multiplying according to a predetermined order, and wherein said plurality of predetermined frequency bands are frequency bands which have a correlation with increasing or decreasing levels of said at least one substance level to be measured; and a data exchange platform in network communication with said user identification module operatively coupled to at least one wearable device of individual users, via said communication network.

In one embodiment, the signal processing module is further configured to evaluate a value from the at least one average power derived for each frequency band. In operation, the value is evaluated by combining the at least one average power for each frequency band by dividing and/or multiplying according to a predetermined order, and the predetermined order is determined based on said at least one substance level.

In one embodiment, the estimation module is further configured to determine values of constant b and C by measuring the average power for the plurality of predetermined frequency bands for at least one power level for each substance to obtain a corresponding at least one value, plot the at least one value for each substance level, select a ratio corresponding said at least three substance levels for each substance providing a straight line as best fit, and fitting a straight line to said plot and deriving values of b and C; wherein said fitting said straight line comprises fitting said at least one substance level as a function of said at least one value. Further, the substance is selected from hormones, neurotransmitters, neuromodulators, biomarkers, EEG digital markers of hormones, neurotransmitters, neuromodulators and biomarkers; and the multiple frequency bands are selected from delta, theta, alpha, beta, SMR, high beta and gamma.

In one embodiment, the user identification module further includes: a determination sub-module configured to determine whether data has been derived for the at least one substance level, an analysis sub-module configured to retrieve data from an EEG database, an EOG database and the data is computed to determine at least one EEG algorithm per substance, and a historical sub-module configured to present the data in a graph format, and/or a report to view historical data and retrieve previous history data over more than one cycle of activity of the at least one substance to project future cycles of the at least one substance level.

In one embodiment, the at least one data parameter is selected from birth gender of said individual users, age, sleep state and sleep stages and the determination of the more than one EEG algorithm per substance is able to determine regression line equation that is provided with a strength of correlation with the substance.

Embodiments of the present invention disclose, a wearable device including a plurality of add-on electrodes for measuring signals from individual users, a configuration module configured to adjust a plurality of frequency bands, markers, impedance and noise filter to optimize recording of at least one EEG signal. In use, the at least one wearable device is operable to authorize sharing of the measured signals with at least one third-party device via a wearable device graphical user interface (GUI).

In accordance with an aspect of the embodiments described herein, a system is provided with a database that is built based on user's EEG data, EOG data and/or response taking into account multiple data parameters.

In one embodiment, an AI algorithm executed by an EEG module is used to predict the selected hormone, biomarker or neurotransmitter on the processed EEG data resulting and returning a numeric value of that hormone, neuromodulator, biomarker or neurotransmitter.

In another embodiment, the plurality of data parameters is used to derive at least one EEG algorithms for cortisol levels and levels of other hormones (ACTH, Testosterone, Estrogen) in the blood.

In yet another embodiment, an analysis module is retrieving data from EEG database and considering one or more data parameters selected from gender and sleep-wake states of an individual to determine more than one EEG algorithm per substance.

As described in WO/2013/008011, the method involved running analyses on a database of EEG data and substance level such as hormone levels, neurotransmitter levels and or the level of biomarkers in the body from any medium. The EEG algorithm derived using this method is found to be statistically significant for each substance.

In one embodiment, the state of consciousness of the individual is determined either by EEG or by selecting/inputting the state of consciousness.

### BRIEF DESCRIPTION OF THE DRAWINGS

FIG. 1 is a flow chart of the method to determine levels of substance(s) from the EEG data and how to improve the accuracy according to one embodiment of the present disclosure;

FIG. 2 is a flow chart of the present method of improving the accuracy to determine at least one substance level in the body of the subject, according to one embodiment of the present disclosure;

FIG. 3 is a flow chart of the present method to measure at least one substance level non-invasively from the EEG according to one embodiment of the present disclosure;

FIG. 4 is a block diagram of the present system, according to one embodiment of the present disclosure;

FIG. 5A is a block diagram of components of a data exchange platform admin, according to one embodiment of the present disclosure;

FIG. 5B, is a block diagram of components of the User Identification Module, according to one embodiment of the present disclosure;

FIG. 6 is a flow diagram of method steps to measure substance levels in the present system in Business to Consumer (B2C) environment, according to one embodiment of the present disclosure;

FIG. 7 is a flow diagram of method steps to measure substance levels in the present system in Business to Business (B2B) environment, according to one embodiment of the present disclosure;

FIG. 8 is a flow diagram of method steps to measure substance levels in the present system with APIs, according to one embodiment of the present disclosure;

FIG. 9A is a plot of the Cortisol level taken from salivary samples of the estimated EEG levels plotted against actual blood levels of Cortisol for each individual at different time intervals, according to one embodiment of the present disclosure;

FIG. 9B is a plot of the ACTH level of the estimated EEG levels plotted against actual blood levels of ACTH for each individual at different time intervals, according to one embodiment of the present disclosure;

FIG. 9C is a plot of estimated EEG levels plotted against actual testosterone level of males at different time intervals, according to one embodiment of the present disclosure;

FIG. 9D is a plot of estimated EEG levels plotted against actual blood level of testosterone in females at different time intervals, according to one embodiment of the present disclosure;

FIG. 9E is a plot of estimated EEG levels plotted against actual estrogen level of males at different time intervals, according to one embodiment of the present disclosure;

FIG. 9F is a plot of estimated EEG levels plotted against actual blood level of estrogen in females at different time intervals, according to one embodiment of the present disclosure;

FIG. 9G is a plot of one day trial testing data of EEG estimated levels plotted against actual blood level of estrogen in a male at different time intervals, according to one embodiment of the present disclosure;

FIG. 9H is a plot of one day trial testing data of EEG estimated levels plotted against actual blood level of testosterone in a male at different time intervals, according to one embodiment of the present disclosure;

FIG. 9I is a plot of one day trial testing data of EEG estimated levels plotted against actual blood level of cortisol in a male at different time intervals, according to one embodiment of the present disclosure;

FIG. 9J is a plot of one day trial testing data of EEG estimated levels plotted against actual blood level of estrogen in a female at different time intervals, according to one embodiment of the present disclosure;

FIG. 9K is a plot of one day trial testing data of EEG estimated levels plotted against actual blood level of testosterone in a female at different time intervals, according to one embodiment of the present disclosure; and

FIG. 9L is a plot of one day trial testing data of EEG estimated levels plotted against actual blood level of cortisol in a female at different time intervals, according to one embodiment of the present disclosure;

### DETAILED DESCRIPTION

### Terminology

AI/ML—Artificial Intelligence/Machine Learning
EEG brainwave activity, typically electroencephalography
EOG eye movement activity, typically electrooculography
The word “subject” refers to human being or animal.
The word “substance” refer to hormones, neurotransmitters, neuromodulators, and or biomarkers.
The use of the word “frequency band” as an essential feature link is about the average power of the frequency band in relation to the levels of substance to determine.
The term “predetermine frequency bands” is/are average power of those frequency bands that correlate with a substance level.
The term “amalgamated ratio” is the ratio that combines the average powers of the predetermined frequency band by multiplying or dividing in a way that result in a value which shows a straight-line of significant correlation with levels of a substance.
The word “gender” considers the biological gender of male and female, research to be carried out for gender neutral.
The term “Sleep-Wake state” refers to the wake state, Sleep State of which there are stages: N1, N2, N3 and REM

Before discussing exemplary embodiments of the present invention in detail, it will be appreciated that the invention may be embodied in a method, a system, and/or in a computer program product. For example, a method of the present invention may be carried out by one or more user's using computers, and a program product of the invention may include computer executable instructions that when executed by one or more computers cause one or more computers to carry out a method of the invention.

A program product of the invention, for example, may include computer code that resides on both a server and a client computer/client computing device, and causes both of the server and client computers to carry out various actions. Further, one or more computer(s) that contains a program product of the invention may embody a system of the invention. It will accordingly be appreciated that in describing a particular embodiment of the present invention, description of other embodiments may also be made. For example, it will be understood that when describing a method of the invention, a system and/or a program product of the invention may likewise be described.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present invention may be written in any combination of one or more programming languages.

Embodiments may also be implemented in cloud computing environments. In this description, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Before describing embodiments of the invention, it will also be appreciated that the present invention will prove beneficial and advantageous when practiced with any of a number of applications where the present invention deriving improved algorithms are deployed to measures hormones and or neurotransmitter levels which are linked to biomarkers to indicate the following and not limited to: well-being, performance status, fertility, mental health, health indicators, R&D, stress and relaxation status of the individual.

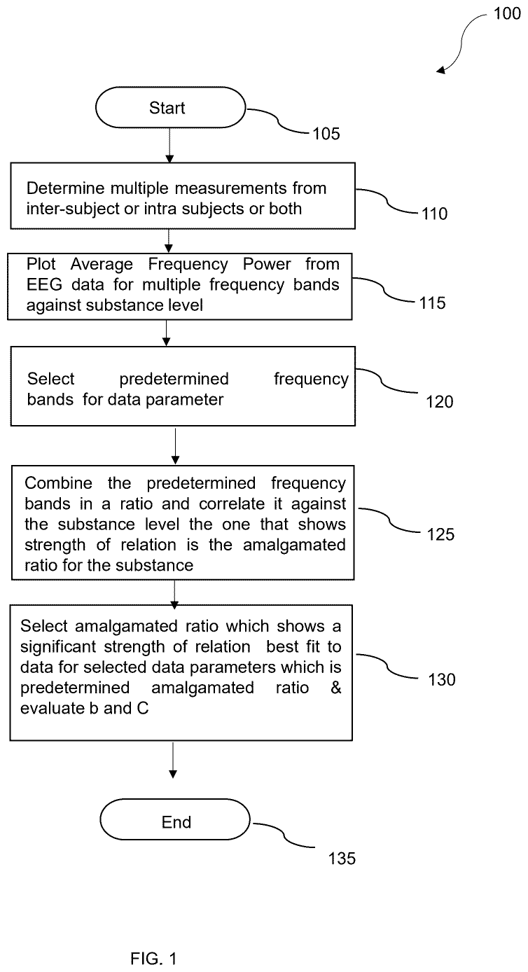

### FIG. 1

FIG. 1 outlines the method for determining these the EEG algorithm to measure substance levels in the body from any medium, in accordance with one or more embodiments of the present invention.

The method 100 starts at Step 110 where multiple data sets of EEG, substance(s) levels for each subject is obtained.

In one embodiment, the substance is selected from ACTH, cortisol, hormone, neurotransmitter, and biomarker. Moreover, the substance is selected from testosterone, estrogen, serotonin, dopamine, thyroxin T3 T4 GABA, Adrenaline, CRH, Noradrenaline, Norephinerine, Acetylcholine, progesterone, LH, insulin, lactic acid, cholesterol, norepinephrine, oxytocin, glutamate, nicotineomide and FSH.

In one embodiment the EEG data is obtained with data parameter: gender, sleep-state and age-group.

In another embodiment, the EEG data and used along with the EOG data to simplify sleep-wake and sleep-stage analysis/state to improve the EEG algorithm for the substance levels depending on the sleep-wake state.

In yet another embodiment the sleep-wake state is determined through a manual entry or is automated in consideration with the EOG to select the EEG algorithm for that substance for that state to improve the accuracy to determine the substance level.

The method 100 proceeds to Step 115. At Step 115, the EEG frequency bands selected from delta, theta, alpha, beta, SMR, high beta and high gamma. Typically, these bands are taking to encompass the frequency ranges: The exact boundaries vary but the general frequency bands are: delta 1-3 Hz; theta 3-7 hz; alpha 7-11 hz; beta 11-25 hz; smr 15-19 hz; high beta 20-30 hz and gamma 35 hz+ are plotted against the levels of the substance for each subject. WO2013008011A1 methods to further improve the accuracy of the essential feature is to correlate the average power of frequency bands into smaller band bins (e.g. 1 Hz bins) with substance levels, to find the precise frequency bands that correlate with the levels of the substance and group the bands that correlate closely together in relation to the substance level, to determine the actual frequency bandwidth thereby deriving the predetermining frequency bands for that substance.

In one embodiment, data is analysed to obtain the average power (UV\hat{ }2/Hz or 10*log 10 UV\hat{ }2/Hz) for each of a plurality of predetermined frequency bands; calculating a value from the average powers derived for each frequency band.

In one embodiment of this present invention the frequency bands are correlated with levels of substances for each subject by any one of the following groups, gender, age and sleep-wake state or by any combination of these groups to improve the accuracy of the EEG algorithm for measuring substance levels.

The method 100 proceeds to Step 120. At Step 120, the frequency band that show a straight line relationship with increasing or decreasing levels of a substance determine the predetermined frequency bands of the substance. The correlation can be improved by slightly varying the boundaries of the predetermined frequency bands. Therefore, the process may be repeated by varying the boundaries on the frequency bands to evaluate a better correlation which would further optimize and improve the algorithm.

The method 100 proceeds to Step 125. At Step 125, pre-determined frequency band is selected for gender, age, sleep-wake state and/or gender type and sleep-awake state.

The method 100 proceeds to Step 130. At Step 130, the average power of the predetermined frequency bands are combined in a ratio by dividing and multiplying in a way that results in correlation with the substance level, the ratio that shows strength of relation is the amalgamated ratio X for that substance, Y.

The method 100 proceeds to Step 135. At Step 135, the regression line equation for the amalgamated ratio and the substance level, derives b and C for the substance Y. where there can be more EEG algorithms per substance are determined as there may be multiple amalgamated ratios that show a significant correlation with the substance resulting a varied regression equations, however the strongest amalgamated correlate would provide the most accurate measure of substance from the EEG data.

In an embodiment of the present invention the regression line equation is plotted against the substance to see if there are any significant errors to include to improve the EEG algorithm, resulting in an Y=bX+C+E

A further embodiment of step 135 an auto regression is applied between the EEG amalgamated ratio X the substance levels to determine improvement of the accuracy of the substance from EEG data as a function of time series analyses.

The auto regression analysis can still be expressed as y=mx+b and a yet a further embodiment where if significant +E

In a further embodiment of the step the equation can be expressed in an auto regression equation for a relationship between the EEG and the substance level based on a function of time.

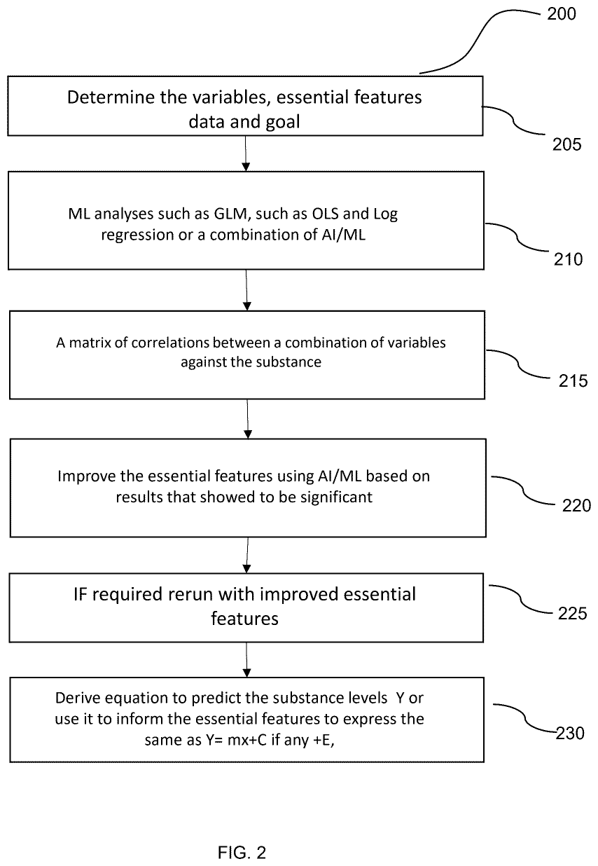

### FIG. 2

FIG. 2 is a flow diagram of the present invention of improving the accuracy and speed of determining the EEG algorithm(s) to measure one or more substance levels illustrated in FIG. 1 method 100 according to one embodiment of the present disclosure.

In one embodiment, the substance is selected from ACTH, cortisol, hormone, neurotransmitter, and biomarker. Moreover, the substance is selected from testosterone, estrogen, serotonin, dopamine, thyroxin T3 T4 GABA, Adrenaline, CRH, Noradrenaline, Norephinerine, Acetylcholine, progesterone, LH, insulin, lactic acid, cholesterol, norepinephrine, oxytocin, glutamate, nicotineomide and FSH.

In the present embodiment the method 200 is to obtain at least 3 data set of EEG data with or without EOG data and substance levels

In another embodiment the EEG dataset for a substance(s) is obtained with further data parameter such as gender, age-group and/or sleep-wake state.

In a further embodiment this data can be obtained from study trial or a database stored on a cloud, server, hard-drive or storage capacity for data access to determine the EEG algorithm to measure levels of substances.

In another embodiment, the EEG data is collected from multiple electrode positions by using any available wearable device.

Step 205 involves determining the essential features such as frequency bands, electrodes of the EEG data for the goal to predict, such as substance levels in this present invention and other data parameters such gender, age-group, or sleep-wake state from inter-subject or intra subjects or both.

In a further embodiment data includes EOG.

In an embodiment method 200 proceed to Step 210 where General Linear Models are applied to carry out and automate the method in FIG. 2 to determine EEG algorithm to measure substance levels. General Linear Models can include Ordinary Least-Squared (OLS), Log Regression are two examples of models that can be applied to return related variable to the substance level and the nature of the variable relate as a model to measure substance levels from the EEG data.

A further embodiment at step 215 applies AI/ML in a series or any combination to carry out steps 115 to steps 135 as correlation matrix between the variables determined in step 205. This allows multiple analyses to be carried to determine the feature variables linked to the substance levels and results in a way to determine the ratio obtained by combining said at least one average power for said each frequency band by dividing and/or multiplying according to a predetermined order; and wherein said plurality of predetermined frequency bands are frequency bands which have a correlation with increasing or decreasing levels of said at least one substance level to be measured.

In one embodiment at FIG. 2, step 220 considers the refinement of step 115 of FIG. 1 of specialising the predetermined frequency bandwidths for the substance by using AI/ML, instead of proceeding with generic bandwidths to derive the predetermined frequencies or essential feature for that substance. An example is to apply a nonlinear model like k-means cluster to determine the natural frequency band widths and therefore determining specific predetermined frequency bands for that substance, which can further improvement accuracy both in the correlate value and in statistical significance.

A further embodiment at step 225 applies a combination of AI/ML that use the essential features of estimating/predicting the substance levels from the EEG data, the electrodes or channels measuring substance levels in the body.

And yet in another embodiment at step 220 the present invention improves and further refines the accuracy to apply AI/MI with essential features, electrodes, gender and any of other data parameter such as sleep-wake state and age-group.

In yet another embodiment from step 220 of this present invention, of not method 100 and 200 consider the variable, essential features such as power of FFT frequency bands and electrodes, groups; such gender, age-group and sleep-wake and sleep stage states, the time windows of the EEG data and the goal such as substance level and added signal data such as EOG along with micro variables further enhancement such as EEG configuration settings such as noise, filters, low and high bandwidths cut-offs, sampling frequency, data resolution and windowing method.

Step 225 of FIG. 2 of the present inventions includes an embodiment where all improvements are applied before setting the variable for the correlation matrix at step 215. This looping back of improvements from steps of method 100 of FIG. 1 and method 200 of FIG. 2 optimizes the accuracy at every step and then run through a correlation matrix to determine the optimized essential features; that is average power of the predetermined frequency band for the substance Y.

Yet a further embodiment is to rerun the optimized outcomes at every step, through a correlation matrix by applying ML General Linear Models on all the variables, to determine step in FIG. 1 step 110 and FIG. 2 step 205 as a loop to find the most accurate EEG algorithm(s) to measure a substance by just substance, gender, age or sleep-wake state.

An embodiment of this invention at step 230 In another embodiment, to further refine the accuracy of the EEG algorithm, the process outcomes of FIGS. 1 and 2 is looped back to the same effect to find the values of b and c of y=bx+c, where y is the substance level to predict and X is the EEG amalgamated ratio for the substance.

Y=bX+C+ε, where the accuracy can be further refined the process is looped back to the same effect to find the values of b and c of y=bx+c+ε, to further refine the accuracy of the EEG algorithm, the process outcomes of FIGS. 1 and 2 is looped back to the same effect to find the values of b and c where y is the substance level to predict, X is the EEG amalgamated ratio for the substance +ε is the error.

In another embodiment at step 230 of present invention, the accuracy of the EEG algorithm is improved by applying auto-regression is often used in time series analyses, in this case it was used to find the EEG data that correlates strongly with the substance of subject over a particular time.

FIGS. 1 and 2 In another embodiment, the present data is based on dominant biological gender and is selected from male, or female. Further, the data is based on the age group of the gender. The information analyses carried out by the data parameter groups impact the accuracy of the present EEG algorithm significantly enough to be statistically significant as an estimate to measure hormones, neurotransmitter, neuromodulators and/or biomarkers.

Running the analyses by gender and sleep-wake states provide significant change and the improved algorithm can distinctly be different between genders for the same substance. Further, running the EEG analyses for a single substance given there is no stark visual difference in the EEG between genders as there are between sleep and wake states further the dynamics of the EEG activity and the brain has much to be explored to be able to infer the substance levels would differ from EEG activity not just across gender, but across a person over the day.

Another parameter of age provides different hormonal stages which is a parameter to explore to what extend the EEG changes. The data was acquired in a study which founded the algorithm for three new substances, testosterone, estrogen and ACTH, however the algorithm varied not just by the combination of predetermined frequency bands but by the parameters, unrelated to processing of the data.

In one embodiment, the EEG data may be acquired from any number of channels and locations from which the associated frequency bands are derived from and used in the calculation to predict hormone levels, neurotransmitter, neuro modulators and/or biomarker.

In an embodiment, at least two signal are acquired for the EEG data. The average power spectrum may be obtained by a Fast Fourier Transform of artefact free EEG data or recording of the acquired EEG data.

In another embodiment, each substance is analysed against the EEG data taken in various time points from the time of substance acquisition to increase the accuracy of EEG algorithm to measure or predict the level of the substance.

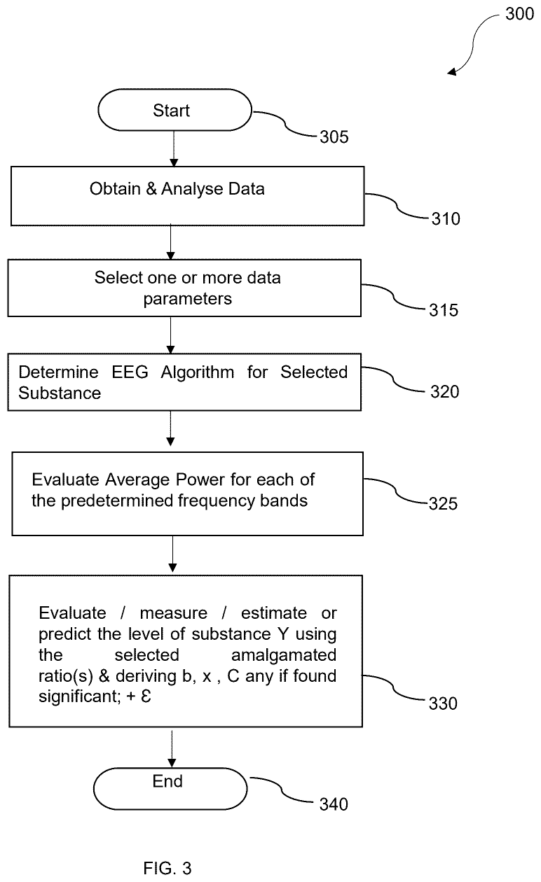

### FIG. 3

FIG. 3 is a flow chart of the present method steps to measure substance levels, in accordance with one or more embodiments of the present invention. The method 300 starts at Step 305 and proceeds to Step 310. At Step 310, an EEG and EOG measurement of the individual is performed and monitored based on the one or more data parameters.

In one embodiment, the EEG data is obtained without considering the EOG data.

In another embodiment, the EEG data is obtained considering the EOG data.

In one embodiment, the data selected is EEG data and/or the EOG data. However, the analysis can take into consideration EEG data with EOG data or EEG data without EOG data. Particularly, the EEG data is analyzed over predetermined frequency bands. This analysis may be performed as the EEG is measured or the analysis may be performed off-line or once recorded and uploaded for a duration of at least 3 seconds. Typically, the predetermined frequency bands are selected from the known bands of delta, theta, alpha, beta, SMR, high beta and gamma. The one or more frequency bands are selected based on the substance which is to be measured.

In one embodiment, the frequency bands are selected in Step 310 from the well-established frequency bands.

The method 300 proceeds to Step 315. At Step 315, the user data parameters are selected for the analysis. The method 300 proceeds to Step 320. At Step 320, the average power for each predetermined frequency band is calculated. Calculation of the average power of the frequency bands are a well-known technique and can be performed using known FFT techniques. Subsequently, a ratio will be determined from the average powers of the predetermine frequency bands calculated at Step 320.

In one embodiment, a value is evaluated from the at least one average power derived for each frequency band, and the value is evaluated by combining the at least one average power for each frequency band by dividing and/or multiplying according to a predetermined order, and wherein said predetermined order is determined based on said at least one substance level.

The method 300 proceeds to Step 325. At Step 325, the EEG algorithm is determined and selected for the substance. The method 300 proceeds to Step 330. At Step 330, the amalgamated ratio is selected based on the data such as gender, age, sleep-wake state of consciousness or both. However, the amalgamated ratio will be different dependent on the order in which the average powers of the predetermined frequency bands are combined to correlate best with the level of substance for each group. Once the order has been determined, this can be saved and looked up when performing method Step 325.

In one embodiment, the substance level is calculated using the amalgamated ratio derived and by using the equation: Y=bX+C or uses Y=bX+C+error if it optimizes the algorithm's accuracy, where b and Care constants which can be looked up for a particular substance. How these constants are derived is described with reference to FIG. 1. X is the amalgamated ratio and Y is the substance level to be determined.

Once these variables have been determined, they are stored in the database of the system 400 and accessed to perform the analysis for a specific substance. The method 300 proceeds to Step 335. At Step 330, the method 300 end.

In yet another embodiment, the data results are used for training the specific frequency or frequencies based on the algorithm.

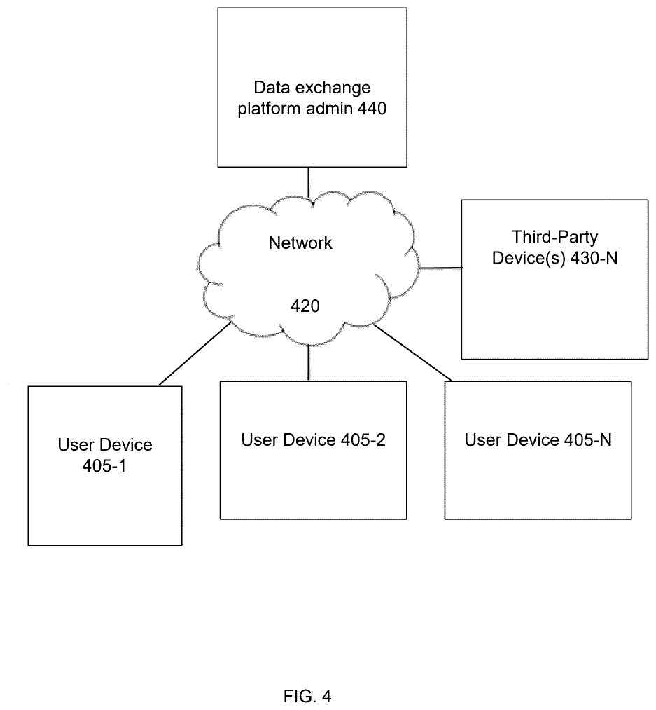

### FIG. 4

FIG. 4 illustrates an example environment in which some exemplary embodiments of the present disclosure may be practiced, according to one or more embodiments of the present disclosure. A system 400 of determining a substance level in the body of the subject from data includes a data exchange platform admin 440, at least one user device 4051, 4052, . . . , 405N, a user identification module 415, an estimation module 435, a communication network 420 for individual users to be connected via the at least one user device 405 and multiple third-party devices 4301, 4302, . . . , 430N. In operation, the data exchange platform admin 440, the one or more user devices 405, multiple third-party devices 4301, 4302, . . . , 430N and at least one remote server 410 in communication with the user identification module 415.

The system 400 is implemented as a cloud server, it may be understood that the system 400 may also be implemented in a variety of user devices, such as but are not limited to, a portable computer, a personal digital assistant, a handheld device, a mobile, a laptop computer, a desktop computer, a notebook, a workstation, a mainframe computer, and the like.

In one embodiment, the network 420 may be a wireless network, a wired network or a combination thereof. The network 420 can be accessed by the device using wired or wireless network connectivity means including updated communications technology.

In one embodiment, the network 420 may be a wireless network, a wired network or a combination thereof. The network 420 can be implemented as one of the different types of networks, cellular communication network, local area network (LAN), wide area network (WAN), the internet, and the like. The network 420 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further, the network 420 may include a variety of network devices, including routers, bridges, servers, computing devices, storage devices, and the like.

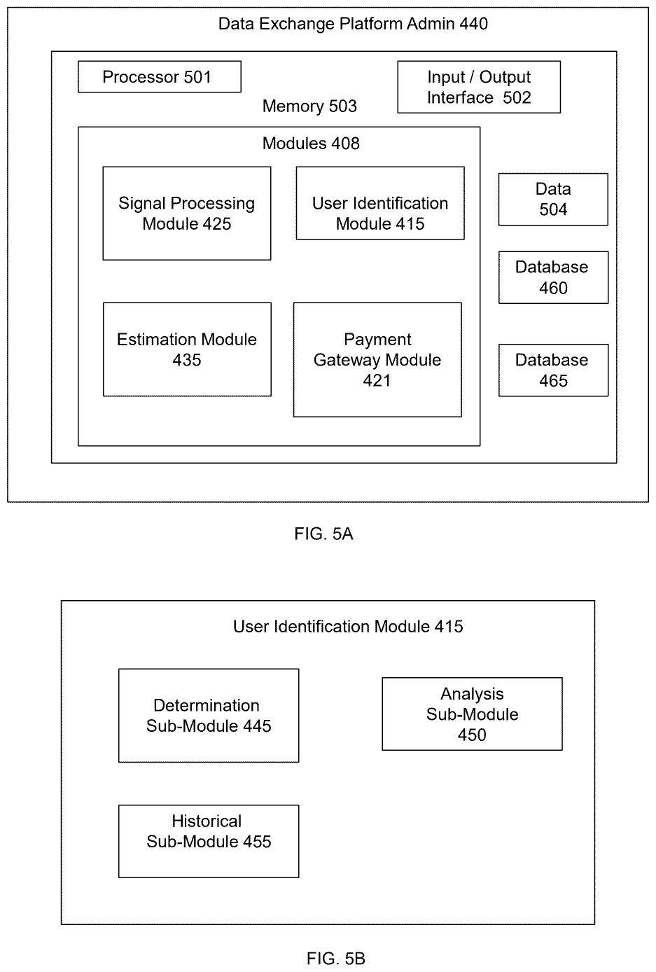

### FIG. 5

Referring to FIG. 5A and FIG. 5B, components of the data exchange platform admin 440, comprises at least one processor 501, an input/output (I/O) interface 502, a memory 503, modules 408 and data 504.

In one embodiment, at least one processor 501 is configured to fetch and execute computer-readable instructions stored in the memory 503. In one embodiment, the I/O interface 502 implemented as a mobile application or a web-based application may include a variety of software and hardware interfaces, for example, a web interface, a graphical user interface, and the like. The I/O interface 502 may allow the admin 440 to interact with the user devices 405. Further, the I/O interface 502 may enable the user device 405 to communicate with other computing devices, such as web servers and external data servers (not shown). The I/O interface 502 can facilitate multiple communications within a wide variety of networks and protocol types, including wired networks, for example, LAN, cable, etc., and wireless networks, such as WLAN, cellular, or satellite. The I/O interface 502 may include one or more ports for connecting to another server. In an exemplary embodiment, the I/O interface 502 is an interaction platform which may provide a connection between users and the admin 440.

In one embodiment, the memory 503 may include any computer-readable medium known in the art including, for example, volatile memory, such as static random-access memory (SRAM) and dynamic random-access memory (DRAM), and/or non-volatile memory, such as read only memory (ROM), erasable programmable ROM, flash memories, hard disks, optical disks, and memory cards. The memory 503 may include modules 408 and data 504.

In one embodiment, the system 400 includes a signal processing module 425 configured to obtain an average power for each of the multiple predetermined frequency bands as mentioned in para

Further, the admin includes signal processing module 425, the estimation module 435, the user identification module 415 and a payment gateway module 421.

In one embodiment, the modules 408 include routines, programs, objects, components, data structures, etc., which perform particular tasks, functions or implement particular abstract data types.

In one embodiment, the system 400 includes at least one remote server (not shown) to process and execute the modules 408 of the data exchange platform admin 440.

In another embodiment, the signal processing module 425 is further configured to evaluate a value from the at least one average power derived for each frequency band. Particularly, the value is evaluated by combining the at least one average power for each frequency band by dividing and/or multiplying according to a predetermined order. The predetermined order is determined based on the at least one substance level. Particularly, one or more user devices 405 are operable to authorize sharing of selected at least one data parameter with a third-party device 430 via a graphical user interface (GUI), thereby creating selected permissible data and the user identification module 415 is operable to exchange the selected permissible data with the third-party device 430.

In one embodiment, the estimation module 435 is configured to obtain an estimate of the at least one substance level from an equation: Y=bX+C, and Y is the at least one substance level to be predicted, X is value and b and C are constants. The value of X is a ratio obtained by combining the at least one average power for each frequency band by dividing and/or multiplying according to a predetermined order. Particularly, multiple predetermined frequency bands are frequency bands which have a correlation with increasing or decreasing levels of the at least one substance level to be measured.

In another embodiment, the estimation module 435 is further configured to obtain an estimate of the at least one substance level from an equation: Y=bX+C+ε, where +ε is the error and Y being the at least one substance level to be predicted, X is value and b and Care constants. The value of X is a ratio obtained by combining the at least one average power for each frequency band by dividing and/or multiplying according to a predetermined order. Particularly, multiple predetermined frequency bands are frequency bands which have a correlation with increasing or decreasing levels of the at least one substance level to be measured. Moreover, the accuracy of the EEG algorithm as mentioned in WO/2013/008011 is improved by including errors (+ε), if the results are statistically significant, resulting in an EEG algorithm of form: Y=bX+C+ε.

In one embodiment, the data exchange platform admin 440 in network communication 420 with the user identification module 415 operatively coupled to any available wearable device(s) of the individual users via the communication network 420.

In one embodiment, the user identification module 415 further includes a determination sub-module 445, an analysis sub-module 450, and a historical sub-module 455. The determination sub-module 445 is configured to determine whether data has been derived for the at least one substance level. Particularly, the analysis sub-module 450 is configured to retrieve data from an EEG database 460, an EOG database 465 and the data is computed to determine at least one EEG algorithm per substance.

In one embodiment, data as contained in EEG feedback is logged and stored in the EEG database 460 for developing the EEG algorithm. Moreover, the at least one EEG algorithm improves and increases accuracy for validating data and predicting at least one substance level from the data.

In one embodiment, EEG data is measured for at least 3 seconds and EEG data is collected from at least EEG signals.

In another embodiment, the EEG data is collected from various electrode positions by using any available wearable device.

In yet another embodiment, the add-on electrodes allows the wearable to be adaptable and movable to acquire signals from the electrode scalp locations based on the substance to measure.

In one embodiment, a historical sub-module 455 is configured to present the data in a graph format, and/or a report to view historical data and retrieve previous history data over more than one cycle of activity of the at least one substance to project future cycles of the at least one substance level. Particularly, the at least one data parameter is selected from birth gender of said individual users, age, sleep state and sleep stages.

In one embodiment, the determination of the more than one EEG algorithm per substance is able to determine regression line equation that is provided with a strength of correlation with the substance. As discussed in the Method 200, the substance is selected from hormones, neurotransmitters, neuromodulators and/or biomarkers, EEG digital markers of hormones, neurotransmitters, neuromodulators and biomarkers.

In one embodiment, the estimation module 435 is further configured to determine values of constant b and C by measuring the average power for the multiple predetermined frequency bands for at least one power level for each substance to obtain a corresponding at least one value, plotting the at least one value for each substance level, selecting a ratio corresponding the at least three substance levels for each substance providing a straight line as best fit, and fitting a straight line to the plot and deriving values of b and C. Particularly, fitting the straight line includes fitting the at least one substance level as a function of the at least one value.

In one embodiment, the one or more remote servers 410 includes multiple distributed servers.

In one embodiment, data 504 include medical history records, real-time sleep data, identity-related data, historical subjective data, historical objective data, etc. For example, but not for limitation, the sleep data includes sleep wake state, total sleep time, time spent in REM, time spent in deep sleep, time to sleep onset, waking time, etc.; and the identity-related data includes age, gender, race, geographical location, etc.

In another embodiment, data is automatically collected and retrieved by the modules 415, 425 of the present system 400 and transmitted by one or more authorized user device 405 via the network communication 420.

In one embodiment, the payment gateway module 421 is in the form of fiat currency, credits, and/or cryptocurrencies. For example, but not limited to, accepted cryptocurrencies include bitcoin, ethers, etc.

In yet another embodiment, the present system 400 provides SDK (Software Development Kit) and APIs (Application Programming Interfaces) for decentralized application program (dApps) development.

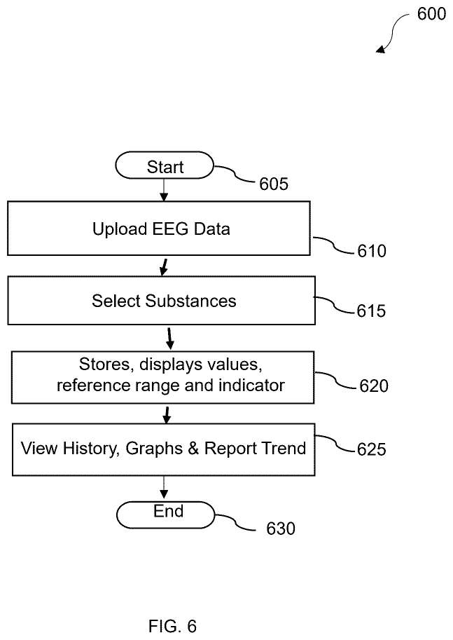

### FIG. 6

FIG. 6 is a flow diagram of method steps to measure substance levels in the present system in Business to Consumer (B2C) environment, according to one embodiment of the present disclosure. The method 600 starts at step 605 and proceeds to step 610. At step 610, the user is able to register in the system 400 and use the different modules of the system 400.

In one embodiment, the EEG is measured using any compatible EEG device. The one or more data parameter is uploaded by the user or streamed using any specific wearable device compatible with the system 400. The method 600 proceeds to step 615. At step 615, one or more substances are selected by the user. The method 600 proceeds to step 620. At step 620, the data is stored in the database of the system 400. In use, the user via the user device 405 is able to retrieve data and value units are displayed via UI. The value may range from references, range and indicator. The method 600 proceeds to step 625. At step 625, the historical trends, graphs are viewed by the user. The report is issued via email. The method 600 proceeds to step 630. At step 630, the method 600 ends.

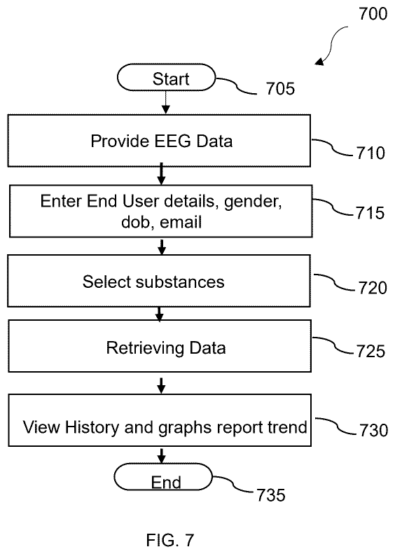

### FIG. 7

FIG. 7 is a flow diagram of method steps to measure substance levels in the present system in Business to Business (B2B) environment, according to one embodiment of the present disclosure. The method 700 starts at step 705 and proceeds to step 710. At step 710, the EEG data is uploaded to the databases of the system 400. The method 700 proceeds to step 715. At step 715, end user details are provided to the system 400. The method 700 proceeds to step 720. At step 720, the one or more substances are selected. The method 700 proceeds to step 725. At step 725, the data is stored in the database of the system 400. In use, the user via the user device is able to retrieve data and value units are displayed via UI. The value may range from references, range and indicator. The method 700 proceeds to step 725. At step 725, the historical trends, graphs are viewed by the user. One or more trend filters are provided to the end user. The results are reported to the end user via email. The method 700 proceeds to step 730. At step 730, the method 700 ends.

In one embodiment, the reference ranges of the EEG data are displayed. The EEG data is able to indicate abnormal and normal levels. One or more modules of the present system are configured to present the data in a graph format, and/or a report to view history by selecting date or date time range (historical data) with the option to download, a calendar UI to enter data that can also project future trends based on previous historical data. Particularly, one or more modules are configured to retrieve previous history data over more than one cycle of activity of a substance to project future cycles of substance levels. Moreover, the EEG data is able to indicate abnormal levels and the normal levels.

In yet another embodiment, the display of results indicates abnormal and normal level or as a representative icon.

In yet another embodiment, the user is provided with the option to select substances or a biomarker where the biomarker can return the results of the substances and which can be used to indicate a biomarker. For e.g., biomarker such as hypothyroid if selected would return measures to store, pass and display for thyroxine, T3, T4 with corresponding indication of abnormal and normal levels based on conditional statements against the corresponding reference range. The ratio of these hormones is an established method to indicate thyroid issues such as hypothyroid or hyperthyroid. This is also looked up and retrieved, stored in the database having data 504 and also displayed on the UI.

In yet another embodiment, the display of results indicates abnormal and normal level or as a representative icon.

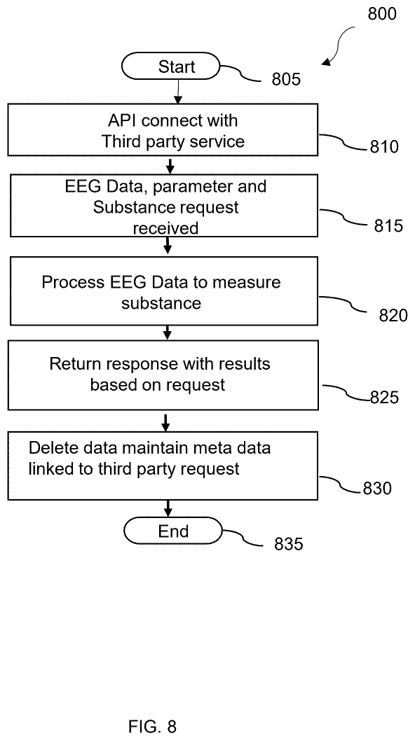

### FIG. 8

FIG. 8 is a flow diagram of method steps to measure substance levels in the present system with APIs, according to one embodiment of the present disclosure. The method 800 starts at step 805 and proceeds to step 810. At step 810, the present system 400 is provided to measure hormones, neurotransmitter, neuromodulators and biomarkers via an Application Programming Interface (API) service connected to third party services. At step 815, the requests are sent with EEG data, if available to the system 400.

In one embodiment, EOG data along with the data parameters selected from the age, gender and age-group of the source of the EEG and the substance(s) to measure are received by the system 400.

At step 820, the EEG data is processed by the one or more modules 408 of the system 400 according to the parameters and substance requested by the user. At step 825, the determined results are returned with units, the reference ranges against senders' request ID. At step 830, the data is deleted in the last step, except for meta data linked to sender's ID and number of queries in the request. At step 835, the method 800 ends.

In one embodiment, the Third-Party service via the third party devices 430 registers with the system 400 to request measure of substance(s) from the EEG data as part of enhancing their own third-party's service.

In a further embodiment, the third-Party service is authenticated prior to importing the API which can be imported.

In another embodiment, the third-party service users purchase a subscription package of credits of measures via the payment gateway module of the present system 400.

The method proceeds activating the API to receive requests as in and send results then delete data.

### Graphs

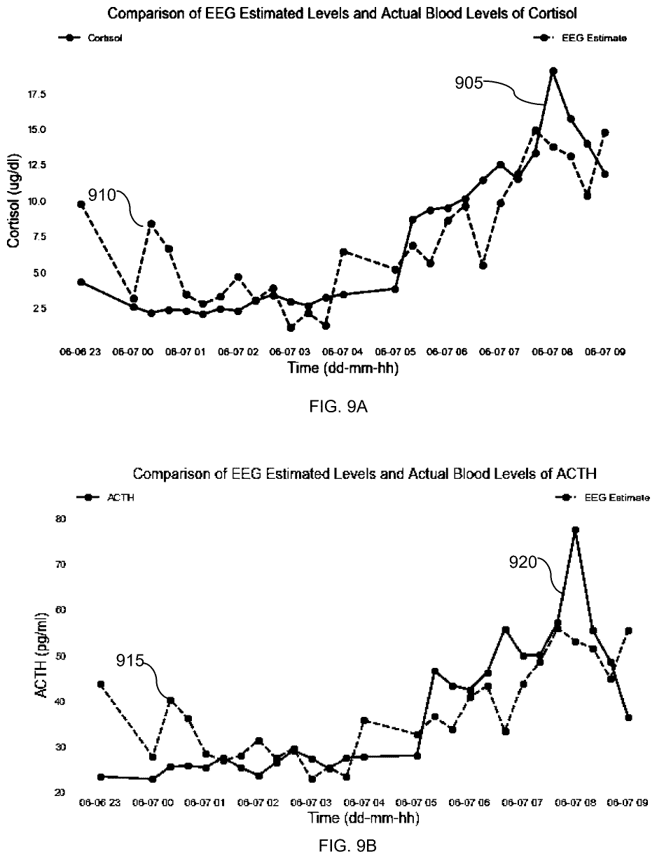

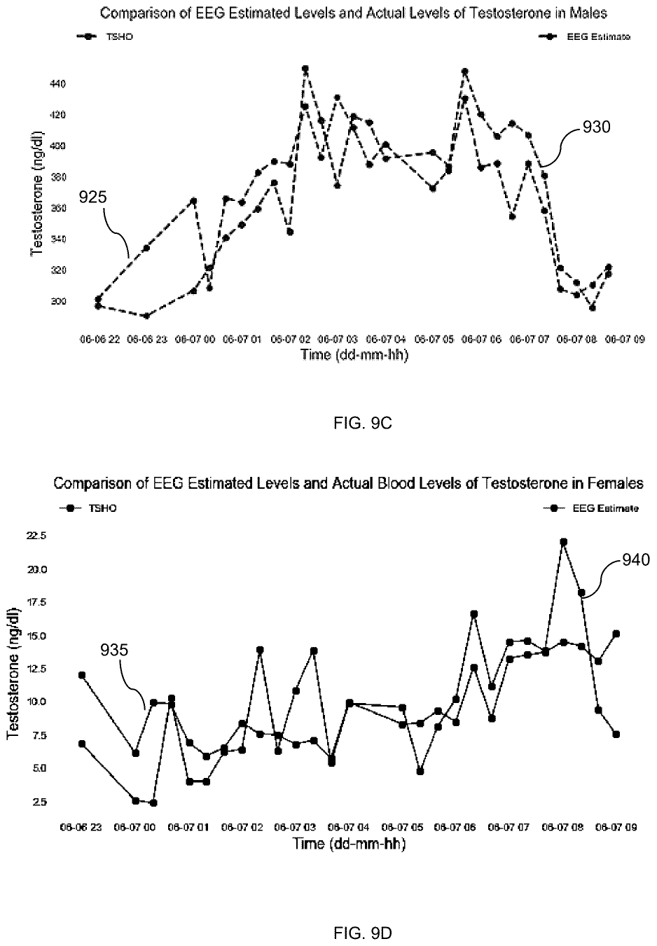

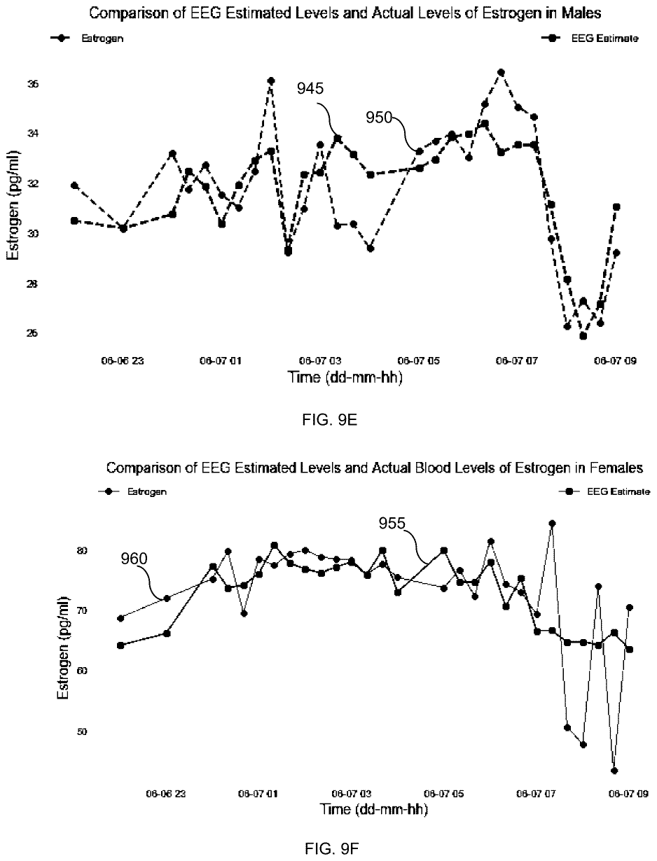

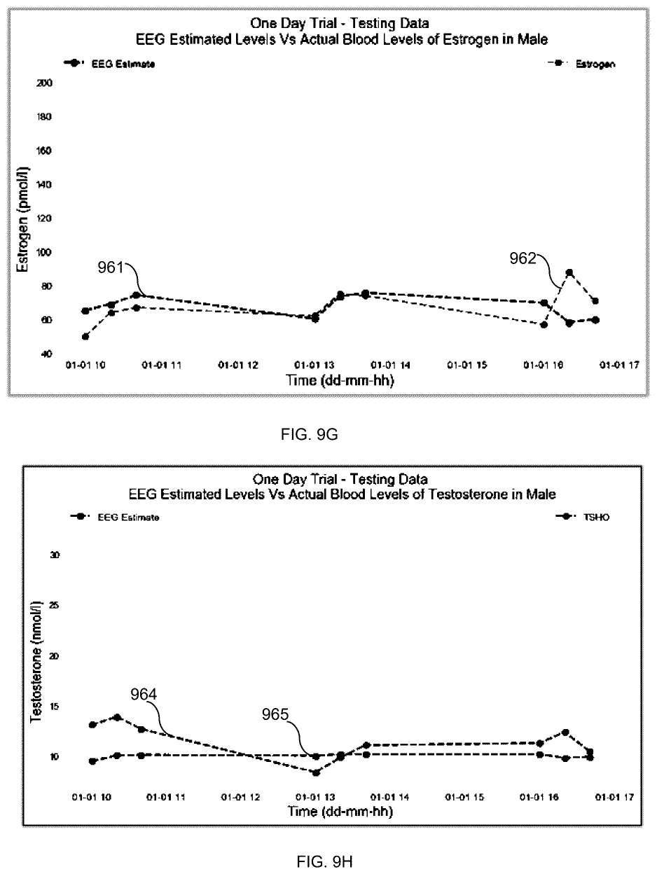

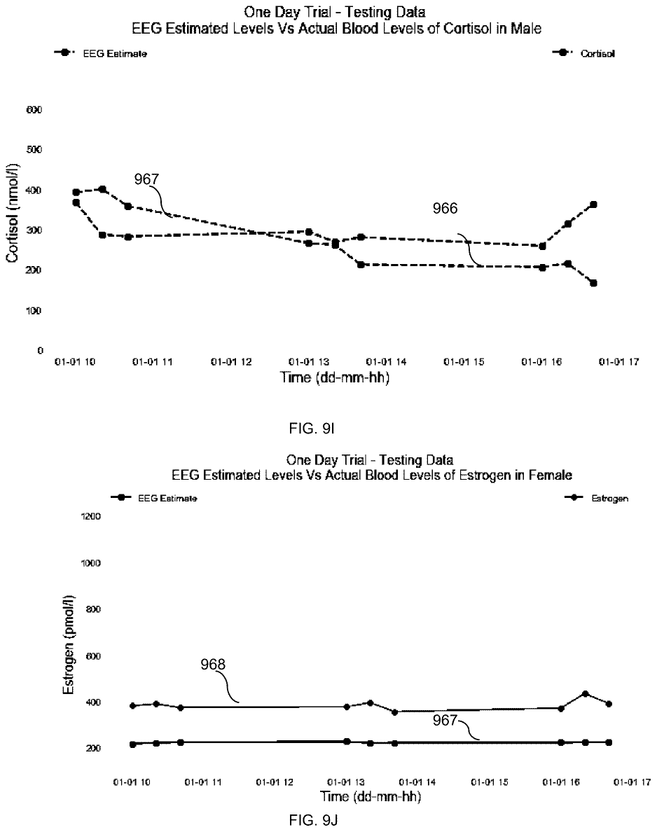

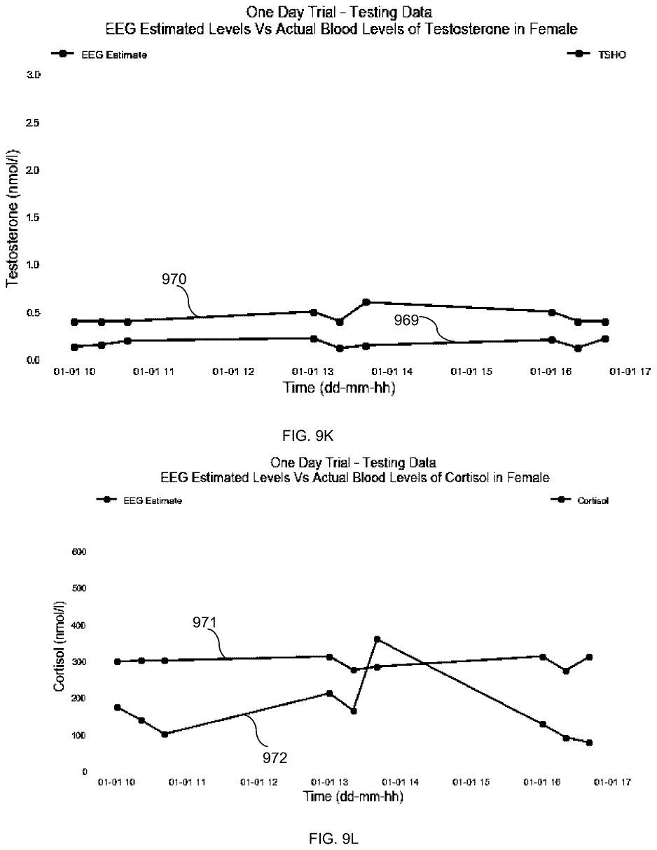

FIG. 9A is a plot of the Cortisol level taken from salivary samples of the estimated EEG levels 910 plotted against actual blood levels of Cortisol 905 for each individual at different time intervals, according to one embodiment of the present disclosure. Pearson's correlation coefficient is highly significant at r=0.81, p=0.0000002 between the estimated EEG levels 910 and actual blood levels of Cortisol 905. In particular, the power relationship of these frequencies as a ratio provides a quantitative measure of salivary Cortisol. For research use or as an applied form of therapy, the effects or efficacy of the therapy can be evaluated by reference to the predicted Cortisol levels.

Cortisol levels are predicted by entering the amalgamated ratio of the frequencies as in the regression line equation:

Where Y=bX+C

Pearson's correlation coefficient, r=0.81.

FIG. 9B. is a plot of the ACTH level of the estimated EEG levels plotted against actual blood levels of ACTH for each individual at different time intervals, according to one embodiment of the present disclosure. Pearson's correlation coefficient is highly significant at r=0.70, p=0.00004 between the estimated EEG levels 915 and actual blood levels of ACTH 920.

To improve the accuracy of determining a level of substance(s) from the EEG data as described in WO/2013/008011, the analyses considered factors such as gender, age and sleep-wake states, resulting in more than one EEG algorithm per substance. The substance is selected from testosterone and estrogen.

FIG. 9C FIG. 4C is a plot of estimated EEG levels plotted against actual testosterone level of males at different time intervals, according to one embodiment of the present disclosure. FIG. 9D is a plot of estimated EEG levels plotted against actual blood level of testosterone in females at different time intervals, according to one embodiment of the present disclosure. The Pearson's correlation coefficient is highly significant at r=0.80, p=0.0000003 between the estimated EEG levels 925 and actual testosterone level of males 930 in FIG. 9C & actual blood level of testosterone in females 940 in FIG. 9D.

FIG. 9E is a plot of estimated EEG levels plotted against actual estrogen level of males at different time intervals, according to one embodiment of the present disclosure. FIG. 4F is a plot of estimated EEG levels plotted against actual blood level of estrogen in females at different time intervals, according to one embodiment of the present disclosure. The Pearson's correlation coefficient is highly significant at r=0.78, p=0.000001 between the estimated EEG levels 945 and actual estrogen level of males 950 in FIG. 9E. The Pearson's correlation coefficient is highly significant at r=0.581, p=0.001 between the estimated EEG levels 955 and actual blood level of estrogen in females 960 in FIG. 9F.

### Testing Data Trial Male Estrogen

### Test of Normality

The differences of mean between EstM and the EstM_EEG, (EstM_Diff) ranges from −1.662<EstM_Diff<29.824 with a mean difference (d̅)=0.183, Standard deviation (6)=13.66. Shapiro-Wilk test of normality=0.9 where ρ>0.05, H0 is not rejected, EstM_Diff follows a normal distribution.

Difference of Mean t-Test

The difference of means test for the two methods: EstM and EstM_EEG (t=0.016, p=0.987 and so at the α=0.05 level of significance H0 is not rejected, supporting the use of EstM_EEG as a reliable measure of EstM.

FIG. 9H is a plot of one day trial testing data of EEG estimated levels 961 plotted against actual blood level of estrogen in a male 962 at different time intervals, according to one embodiment of the present disclosure.

### Testing Data Trial Male Testosterone

### Test of Normality

The differences of mean between TestM and the TestM_EEG, (TestM_Diff) ranges from −1.662<TestM_Diff<3.827 with a mean difference (d̅)=1.451, Standard deviation (6)=1.826. Shapiro-Wilk test of normality=0.956 where ρ>0.05, H0 is not rejected, TestM_Diff follows a normal distribution.

Difference of Mean t-Test

The difference of means test for the two methods: TestM and TestM_EEG (t=0.839, p=0.047 and so at α=0.05 level of significance, since p value=0.05 on rounding H0 is rejected, supporting the use of TestM_EEG as a reliable measure of TestM.

FIG. 9I is a plot of one day trial testing data of EEG estimated levels 966 plotted against actual blood level of cortisol in a male 967 at different time intervals, according to one embodiment of the present disclosure.

### Testing Data Trial Male Cortisol

### Test of Normality

The differences of mean between CorM and the CorM_EEG, (CorM_Diff) ranges from 110.659<CorM_Diff<343.548 with a mean difference (d̅)=206.141, Standard deviation (6)=71.955. Shapiro-Wilk test of normality=0.962 where ρ>0.818, H0 is not rejected, CorM_Diff follows a normal distribution.

Difference of Mean t-Test

The difference of means test for the two methods: CorM and CorM_EEG (t=0.2366, ρ=0.00 and so at α=0.05 level of significance H0 is rejected, suggesting the use of CorM_EEG is not a reliable measure of CorM.

### Testing Data Trial Female Estrogen

FIG. 9J is a plot of one day trial testing data of EEG estimated levels 967 plotted against actual blood level of estrogen in a female 968 at different time intervals, according to one embodiment of the present disclosure.

### Test of Normality

The differences of mean between EstF and the EstF_EEG, (EstF_Diff) ranges from 148.108<EstF_Diff<227.946 with a mean difference (d̅)=177.516, Standard deviation (6)=22.835. Shapiro-Wilk test of normality=0.905 where ρ>0.28, H0 is not rejected, EstF_Diff follows a normal distribution.

Difference of Mean t-Test

The difference of mean test for the two methods: EstF and EstF_EEG (t=8.122, ρ=0.00 and so at α=0.05 level of significance H0 is rejected, suggesting the use of EstF_EEG is not a reliable measure of EstF.

FIG. 9K is a plot of one day trial testing data of EEG estimated levels 969 plotted against actual blood level of testosterone in a female 970 at different time intervals, according to one embodiment of the present disclosure.

### Testing Data Trial Female Testosterone

### Test of Normality

The differences of mean between TestF and the TestF_EEG, (TestF_Diff) ranges from 0.141<TestF_Diff<0.409 with a mean difference (d̅)=0.231, Standard deviation (6)=0.075. Shapiro-Wilk test of normality=0.822 where ρ>0.036, H0 is rejected, TestF_Diff does not follow a normal distribution.

Difference of Mean t-Test

The difference of means test for the two methods: TestF and TestF_EEG (t=3.186, p=0.00 and so at the α=0.05 level of significance H0 is rejected, suggesting the use of TestF_EEG is not a reliable measure of TestF

FIG. 9L is a plot of one day trial testing data of EEG estimated levels 971 plotted against actual blood level of cortisol in a female 972 at different time intervals, according to one embodiment of the present disclosure.

### Testing Data Trial Female Cortisol

### Test of Normality for Female-Cortisol

The differences of mean between CorF and the CorF_EEG, (CorF_Diff) ranges from −17.879<CorF_Diff<343.744 with a mean difference (d̅)=120.162, Standard deviation (6)=106.295. Shapiro-Wilk test of normality=0.932 where ρ>0.496, H0 is not rejected, CorF_Diff follows a normal distribution.

Difference of Mean t-Test

The difference of means tests for the two methods: CorM and CorM_EEG (t=1.402, ρ=0.003 and so at α=0.05 level of significance H0 is rejected, suggesting the use of CorM_EEG is not a reliable measure of CorM.

| | | TP | TN | FP | FN | |
|---|---|---|---|---|---|---|
| ACC = | Cortisol | | 14 | | 4 | |
| | Estrogen | 9 | 9 | | | |
| | Testosterone | | 18 | | | |
| | All True | 9 | 41 | | | = 50 = 0.925926, 93% |
| | Total | 9 | 41 | 0 | 4 | = 54 |
| SE = | True Abnormal | 9 | | | | = 9 = 1, 100% |
| | Total Abnormal | 9 | | | | = 9 |
| SP = | True Normal | | 41 | | | = 41 = 1, 100% |
| | Total Normal | | 41 | 0 | | = 41 |

Table 1 above calculates Accuracy (ACC)=93%, Sensitivity (SE)=69% & Specificity (SP)=100% of the substances (cortisol, estrogen and testosterone) by comparing the EEG value data against the values received from the blood sample.

The blood value determines if the levels are normal or not based on reference range determined for each substance. In use, if an individual data is in the normal range it would be negative and if abnormal it would be positive. Test is performed to determine if the EEG based values show the same as illustrated below.

TP stands for True Positive when blood is positive and EEG is positive. TN stands for True Negative when blood in negative and EEG based value is also Negative. FP stands for False Positive when blood is negative and EEG based value shows positive. FN stands for False Negative when Blood is Positive and EEG based value is Negative.

In one embodiment, the present modules of the invention are integrated inside a headset to perform the present method steps of the invention.

The present invention is able to improve the accuracy of measuring levels of substance(s) from the EEG data by taking into account data parameters. The data parameters are selected from gender, age, sleep-wake and or gender type and sleep-wake states, resulting in more than one EEG algorithm per substance.

The foregoing only describes one example of the invention, and modifications which are obvious to those skilled in the art may be made thereto without departing from the scope of the invention as defined in the accompanying claims. For example, the general methodology of this invention maybe applied to measuring or predicting the levels of other hormones such as testosterone, progesterone, estrogen, thyroxine, t3, t4, Cortisol and neurotransmitters in the blood, urine or saliva, where the method is the same with three different factors: The location of acquiring EEG activity, it could be from a single or multiple locations of the scalp, the regression equation based on a amalgamated ratio derived from the power of the associated frequencies that is correlated with the substance of measure.

1. A method improving the accuracy to measure levels of at least one substance
using the EEG and substance level data sampled from any medium such as, but not limited to blood, urine and saliva, comprising the steps of:
a. determining the predetermined frequency bands for said substance by at least one data parameter of biological gender, age, sleep-wake state and sleep-stage;
b. determining the amalgamated ratio from the predetermined frequency bands and how they combine to show a significant correlation for said substance;
c. comparing results that are statistically significant and setting at least one amalgamated ration (X), where the amalgamated ratio is determined by combining the predetermined frequency bands in a way that shows at least one statistically significant correlation with the substance level to obtain an improved measure of level of said substance from γ=bX+C;
wherein said at least one substance level (Y) is selected from hormones, neurotransmitters, neuromodulators and/or biomarkers.

2. A method of claim 1 further comprising of:
a. obtaining a measure of levels of at least one substance from an equation: Y=bX+C+ε, and wherein Y is said at least one substance level to be measured, X is the amalgamated ratio value and be and C are constants and +ε is an error;
b. applying auto-regression analyses on determining γ of γ=bX+C or γ=bX+C+ε for said substance;
c. selecting at least one regression equation showing improvement in accuracy;
wherein said substance level (γ) is selected from hormones, neurotransmitters, neuromodulators and/or biomarkers.

3. The method as in any preceding claims, wherein said method further comprises the steps of:
determining predetermined frequency bands for levels of said substance by at least one data parameter of gender, age, sleep-wake state and sleep stage that includes processing the EEG data
to measure the average power for said plurality of frequency bands from at least tow EEG signals or one
a. active channel and selecting at least one band which show significant correlation with increasing or decreasing levels of said substance;
b. combining average powers of predetermined frequency bands of corresponding substance in a way that shows a correlation with levels of said substance to derive the amalgamated ratio;
c. using OLS, plotting, general linear regression or correlation matrix at each preceding step to determine the predetermined frequence bands, the amalgamated ratio(s) and the general linear equation of said substance;
d. using AI for further optional enhancement of accuracy to find the cluster of the average power of frequency bands against the substance to determine predetermined frequency bands that are specialised for that substance;
e. investigate optimising accuracy at each step including auto-regression and looping back into preceding claims as starting variables.

4. A software service system to measure cycle(s) of at least one substance from the EEG data of a user comprising of:
a. receiving EEG data of at least tow EEG signals or one active channel of user;
b. processing the values of the predetermined frequence bands for said substance and biological gender;
c. calculating the amalgamated ratio from the predetermined frequency bands for said substance and gender;
d. obtain a measure from the regression equation for said substance for at least one data parameter from biological gender, sleep-wake stat and sleep-stage, wherein said at least one substance level (Y) is selected from hormones, neurotransmitters, neuromodulators and/or biomarkers.

5. A system of claim 4 further comprises options to:
a. receive at least EEG of EEG and EOG data from file storage;
b. receive at least EEG of EEG and EOG data from connected recording devices to the software service;
c. receive via at least EEG of EEG, EOG an API of third-party service to the software service;
d. determine data parameters of biological gender, age, sleep-wake state and sleep stage from a manual entry is no auto-detectable feature via EEG and EOG data.

6. A system of claim 5 measuring level(s) of at least one substance from user data in a regulatory network management, said system comprising:
a. at least one user device and at least one remote server in communication with a user identification module;
b. a communication network for individual users to be connected via said at least one user device;
c. a data exchange platform admin in network communication with said user identification module operatively coupled to at least one wearable device of individual users via said communication network;
d. user identification module configured to exchange selected permissible data with a plurality of third-party devices;
e. a signal processing module to obtain an average power for the predetermine frequency bands of said substance;
f. seamless integration with a software service system to measure hormones, neurotransmitter, neuromodulators and biomarkers;
wherein, said at least one user device is operable to authorize sharing of selected at least one data parameter with each third-party device via a graphical user interface (GUI), thereby creating said selected permissible data.

7. The system of claims 4 and 5 wherein said user identification module further comprising:
a. a payment gateway module;
b. a determination sub-module configured to determine whether user data has been derived for said at least one substance level which links to the payment gateway module;
c. an analysis sub-module configured to retrieve user data from an EEG database, an EOG database and said user data is computed to select at least one EEG algorithm per substance;
d. a historical sub-module configured to present said user data in a graph format, and/or a report to view historical data, reference ranges and accuracy for each substance measured as per filter options;
e. a feature option to email selected reports;
f. a feature option to view biomarkers based on at least one level of at least one substance derived from using said system;
g. an option for B2B and B2C version of the said software service system.

8. A computer program comprising computer-readable instructions configured to perform and automate the any or all methods of claims 1 to 3.
